(19) United States
(12) Patent Application Publication
(10) Pub. No.: US 2023/0165727 A1
(43) Pub. Date: Jun. 1, 2023

(54) UTERINE HEMOSTASIS DEVICE AND RELATED METHODS OF USE

(71) Applicant: Davol Inc., Warwick, RI (US)

(72) Inventors: Maleeha Mashiatulla, Glen Ellyn, IL (US); Amelia Smith, Rochester, MI (US); Melanie Strimas, Apex, NC (US); Scott Andras, Little Elm, TX (US)

(21) Appl. No.: 17/539,287

(22) Filed: Dec. 1, 2021

(51) Int. Cl. A61F 13/20; A61F 13/26; A61F 13/34

(52) U.S. Cl. CPC A61F 13/2002; A61F 13/2028; A61F 13/2071; A61F 13/2051; A61F 13/2074; A61F 13/266; A61F 13/34; A61F 2013/1517

(57) ABSTRACT

Uterine hemostasis devices and related methods are generally described. In some embodiments, a uterine hemostasis device may include a pouch enclosing one or more fluid absorbing bodies for treatment of postpartum hemorrhage. The pouch may be delivered to a patient's uterus, whereby the fluid absorbing bodies may absorb uterine fluids (e.g., blood) and expand to apply pressure to the uterine walls. Following hemostasis of the uterus, the pouch may be extracted with retrieval tethers accessible from the patient's vagina or vaginal opening. In some embodiments, the pouch may be compressed prior to extraction, such that it may pass through the cervical canal more easily. In some embodiments, the pouch may enclose one or more pouches, such that the fluid absorbing bodies may be extracted in batches to facilitate removal. The pouch may be delivered with a syringe-like applicator positioned in the cervical canal.

### FIELD

Disclosed embodiments are related to hemostatic devices and related methods of use. More specifically, methods and apparatuses related to uterine hemostasis devices for management of postpartum hemorrhaging are disclosed.

### BACKGROUND

Postpartum hemorrhage is one of the top five causes of maternal mortality worldwide, and can be caused by a variety of conditions such as uterine atony (ineffective contraction of the uterus after delivery), uterine tearing, and improper placenta delivery. Symptoms of postpartum hemorrhage include uncontrolled uterine bleeding, decreased blood pressure, increased heart rates, decrease in red blood cell count, hypotension, and in severe instances, death.

### SUMMARY

In some aspects, uterine hemostasis devices are provided. In some embodiments, a uterine hemostasis device includes at least one pouch enclosing at least one fluid absorbing body, wherein the at least one fluid absorbing body is configured to expand in volume upon absorption of a fluid, and at least one tether operatively coupled to an internal surface of the at least one pouch, wherein the at least one tether is configured to be manipulated, and wherein manipulation of the at least one tether at least partially compresses the at least one fluid absorbing body.

In some aspects, uterine hemostasis devices are provided. In some embodiments, a uterine hemostasis device includes a first pouch containing a first at least one fluid absorbing body, a second pouch containing a second at least one fluid absorbing body, the second pouch disposed at least partially in the first pouch, a first tether operatively coupled to the first pouch, and a second tether operatively coupled to the second pouch.

In some aspects, methods of operating a uterine hemostasis device are provided. In some embodiments, a method of operating a uterine hemostasis device includes positioning a first pouch in a uterus of a patient, the first pouch containing a first at least one fluid absorbing body, absorbing fluids from the uterus of the patient with the first at least one fluid absorbing body, and manipulating a first tether to at least partially compress the first at least one fluid absorbing body, wherein the first tether is coupled to an internal surface of the first pouch.

In some aspects, methods of operating a uterine hemostasis device are provided. In some embodiments, a method of operating a uterine hemostasis device includes positioning a first pouch and a second pouch in a uterus of a patient, the first pouch containing a first at least one fluid absorbing body and the second pouch containing a second at least one fluid absorbing body, wherein the second pouch is disposed at least partially within the first pouch, absorbing fluids from the uterus of the patient with the first at least one fluid absorbing body and the second at least one fluid absorbing body, extracting the second pouch from the first pouch and from the uterus of the patient, and extracting the first pouch from the uterus of the patient.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1-2 schematically illustrate a uterine hemostasis device in use, according to some embodiments;

FIG. 3 schematically illustrates a uterine hemostasis device according to some embodiments;

FIG. 4 schematically illustrates the uterine hemostasis device of FIG. 3 in a deployed configuration, according to some embodiments;

FIG. 5 schematically illustrates the uterine hemostasis device of FIG. 3 in a removal configuration, according to some embodiments;

FIG. 6 schematically illustrates a uterine hemostasis device according to other embodiments;

FIG. 7 schematically illustrates the uterine hemostasis device of FIG. 6 in a deployed configuration, according to some embodiments; and

FIGS. 8A-8B schematically illustrate the uterine hemostasis device of FIG. 6 in a removal configuration, according to some embodiments.

### DETAILED DESCRIPTION

Conventional treatment for postpartum hemorrhaging can include medicine or physical massages to induce uterine contraction. In some instances, a flexible vessel, such as a balloon and/or a liquid-expandable packing material (e.g., gauze), may be inserted into the uterus to apply pressure to the uterine walls. These exemplary structures may serve as intrauterine tamponades, such that the pressure applied to the uterine walls may stop or limit bleeding to achieve hemostasis. Conventionally, the packing material (which may comprise a plurality of components, such as rolls of gauze) is manually inserted in the uterus to induce hemostasis, and may therefore need to be removed from the uterus after use to reduce the risk of infection or adhesion formation.

Thus, the inventors have recognized that the manual nature of the conventional techniques for uterine hemostasis may be time and skill intensive. For example, significant training may be required to pack the uterus with the sufficient amount of packing materials to apply an appropriate amount of pressure to the uterus. In addition, manual packing and unpacking of the uterus with individual packing material pieces may pose a risk of infection, adhesion formation, and/or a variety of other adverse reactions if even a single piece is left inside the uterus. The unpacking process typically involves forceps inserted into the vagina/cervix/uterus to grasp and remove the packing materials, which may be invasive for the patient and time-consuming for the clinician.

Based on the foregoing, the inventors have recognized the benefits associated with a uterine hemostasis device for reducing the risk of postpartum hemorrhage. The uterine hemostasis device may be easy to use, such that it may be employed by clinicians without significant training and experience. The uterine hemostasis device may allow packing material to be deployed into the uterus with minimal delivery steps, such that the device may be positioned within the uterus more quickly and/or more easily than with conventional uterine gauze packing with forceps. The system may also permit the packing material to be more quickly, more easily removed, and/or more completely removed (e.g., no materials remaining in the uterus) after the hemostasis procedure, potentially reducing the risk of infection, adhesion formation, and/or other adverse reactions.

In some embodiments, a uterine hemostasis device described herein may include at least one pouch configured to be positioned at least partially in a patient's uterus, the pouch filled with one or more fluid absorbing bodies. The fluid absorbing bodies may absorb uterine fluids (e.g., blood) and expand, thereby applying pressure to the uterine walls to provide a hemostatic effect. The pouch may enclose the fluid absorbing bodies, such that the fluid absorbing bodies may be delivered to and extracted from the uterus simultaneously with the pouch, which may simplify the procedure for the clinician, in terms of both time and skill. In some embodiments, the pouch enclosure may help ensure that every piece of fluid absorbing body has been appropriately extracted from the uterus. In this way, the risk of infection from a misplaced fluid absorbing body (e.g., remaining in the uterus) may be significantly reduced.

In some embodiments, the pouch size may expand upon exposure of the fluid absorbing bodies to the uterine fluids. In other words, the pouch may stretch or otherwise expand to accommodate the expanding fluid absorbing bodies. As such, the pouch may at least partially fill the uterine cavity during the hemostasis procedure. The inventors have recognized that this expanded size of the pouch may be larger than a cross-section of the patient's cervical canal, such that removal of the expanded pouch from the uterus may be challenging. Accordingly, the pouches of the present disclosure include one or more compression tethers, accessible to the clinician from the vagina and/or outside the vaginal opening, configured to partially compress the pouch and reduce its volume (e.g., by partially expelling some of the absorbed blood). In this way, the pouch may be more readily extracted from the patient's cervical canal. Specific implementation and use of the compression tethers are described in greater detail below with reference to the figures.

In some embodiments, the uterine hemostasis devices described herein may include more than one pouch (e.g., a secondary pouch) containing one or more fluid absorbing bodies. The secondary pouch(es) may be positioned inside a main pouch, and may be configured to be extracted independently from the main pouch. The secondary pouch may reduce the overall volume of the main pouch, facilitating removal through the cervical canal. In such embodiments, the secondary pouch may not be as flexible as the main pouch, such that it may not expand as much as the main pouch (e.g., throughout the uterus), and may accordingly be extracted through the cervical canal more easily.

The pouches of the uterine hemostasis devices described herein may be formed of any suitable material which may be fluid permeable. In other words, the pouch may be formed of a material which may not inhibit fluid flow into the pouch. For example, the pouch may be porous. In this way, the fluid absorbing bodies may be able to absorb the uterine fluids (e.g., blood) without exiting the pouch(es). The pouch may be formed of a mesh material (e.g., open worked mesh), with sufficiently large pores to allow fluid to pass through uninhibited. In some embodiments, the pouch may be formed of a fluid-permeable material. In some embodiments, the mesh material may be flexible and/or stretchable. The pouches may be formed of any suitable fluid permeable form factor, including, but not limited to fabrics, meshes, gauzes, sponges, nonwoven fabrics, melt-blown webs, foams, spun-bonded webs, thermal-bonded webs, spun-laced webs, paper, thermally-embossed nonwoven fabrics, woven fabrics, knitted fabrics, non-woven fabrics, perforated films, combinations thereof, and/or any other suitable form factor. It should be appreciated that the pore size of the pouches may permit fluid flow but may inhibit passage of the fluid absorbing bodies out of the pouches—either in the initial un-expanded state (e.g., before absorption of blood), or in the expanded state (e.g., after absorption of blood).

As will be described in greater detail below, the pouch(es) may include one or more retrieval tethers positioned in the vagina and/or outside the vaginal opening, such that a clinician may readily access and pull (or otherwise manipulate) the tethers to extract the pouch(es) from the uterus. The retrieval tethers may be significantly less invasive than conventional means of packing material removal, which involves forceps and repeated re-entry of said forceps inside the patient's uterus. Of course, other functions and/or benefits of the tethers are also contemplated, as the present disclosure is not so limited.

In some embodiments, the uterine hemostasis device described herein may be delivered with an applicator, which may be in the form of a syringe. The applicator may include a barrel, which may hold the fluid absorbing bodies enclosed in one or more pouches, as well as a plunger. The plunger may include at least one flange accessible to the clinician, serving as a push surface, and at least one flange positioned inside the barrel, to urge the pouch(es) out of the barrel. In some embodiments, the flange accessible to the clinician may be located external to the patient (e.g., from outside the vagina), whereas in other embodiments, the flange may be accessible to the clinician internal to the patient (e.g., from inside the vagina). In some embodiments, the plunger may be hollow to allow the one or more tethers to extend from the plunger into the vagina during operation. Of course, non-hollow (e.g., solid) embodiments of the plunger are also contemplated.

In operation, a clinician may insert an applicator into a patient's vagina and press the applicator upwards into the cervical canal. The applicator may be sized to fit within the cervical canal, such that the clinician may be able to determine when to stop (e.g., not pass into the uterus) with a tactile feel. The clinician may then deploy the pouch (which may include the fluid absorbing bodies) by advancing the plunger through the barrel. After the pouch is deployed out of the barrel, the clinician may remove the applicator from the patient. In some embodiments, the position of at least one flange on the plunger may be designed to reduce the risk of the clinician inserting the applicator too far and/or advancing the plunger too far. In some embodiments, the flange may be sized to reduce the risk of the pouch contacting the uterine walls during deployment.

The pouch (which may, in some embodiments, enclose one or more pouches) may be deployed into the uterus, such that the fluid absorbing bodies may begin absorbing fluids from the uterus (e.g., blood) and expanding to apply pressure to the uterine walls. This pressure may serve a tamponading effect and reduce the risk of postpartum hemorrhage. The pouch and fluid absorbing bodies may be left in the uterus for any clinically relevant duration. The clinician may determine that hemostasis has been achieved by measuring the patient's blood pressure and/or any other indicator of hemostasis. Once an appropriate level of hemostasis has been achieved (and/or if the clinician determines that more fluid absorbing bodies are needed), the system may be extracted from the patient. In some embodiments, the pouch(es) may first be partially compressed, expelling some of the absorbed blood, and subsequently extracted through the vagina. The compression may serve to reduce the size of the pouch(es), such that they may pass through the cervical canal more easily. In some embodiments, the system may be extracted in sequence, such that multiple pouches may be extracted sequentially, to reduce the total volume of fluid absorbing bodies that need to pass through the cervical canal. The one or more pouches may be extracted by tethers accessible to the clinician.

In some embodiments, the uterine hemostasis devices may be delivered to the patient without an applicator, for example, with a catheter, although other embodiments of delivery are also contemplated. In some embodiments, the uterine hemostasis device may be directly delivered manually by a clinician, who may insert the pouch (which may be initially small, not having absorbed any uterine fluids) through the vagina and into the cervical canal.

In some embodiments, the fluid absorbing bodies may be formed of any suitable material which may absorb fluids and expand to apply pressure to the uterus. Of course, non-expanding embodiments of the fluid absorbing bodies are also contemplated. The fluid absorbing bodies may be any suitable porous form factor, including, not limited to, fabrics, meshes, gauzes, sponges, nonwoven fabrics, melt-blown webs, foams, spun-bonded webs, thermal-bonded webs, spun-laced webs, paper, thermally-embossed nonwoven fabrics, woven fabrics, knitted fabrics, non-woven fabrics, perforated films, combinations thereof, and/or any other suitable fluid absorbing material.

In some embodiments, the fluid absorbing bodies (and/or any other component of the uterine hemostasis devices described herein) may include a hemostatic agent. The agent may be formed integrally with the fluid absorbing bodies and/or may be a coating on the fluid absorbing bodies. The hemostatic agent may be any composition or combination of compositions which may induce hemostasis (and/or coagulation) known in the art, including, but not limited to, tranexamic acid, calcium chloride, thrombin, glucosamine, chitosan, kaolin, fibrinogen/fibrin, chitin, cellulose, gelatin, alginate, protamine sulfate, and/or combinations thereof. It should be appreciated that in some embodiments, the hemostatic agent may be delivered to the uterus along with a secondary active material, including, but not limited to, antibacterial, antifungal, anti-inflammatory, analgesics, antihistamines, antibiotics, and/or any other active material or combination of materials.

In some embodiments, the fluid absorbing bodies and/or pouches which enclose the fluid absorbing bodies may be formed of a porous, non-bioabsorbable material. Examples of non-bioabsorbable materials include, but are not limited by, polyethylene terephthalate (PET), polyamides, aramids, expanded polytetrafluoroethylene, polyurethane, polyvinylidene difluoride (PVDF), polybutyl esters, polyetheretherketone (PEEK), polyolefins (such as polyethylene or polypropylene), cotton, cellulose, paper, and/or combinations thereof.

In some embodiments, the fluid absorbing bodies and/or pouches which enclose the fluid absorbing bodies may be formed of a porous, bioabsorbable material. In this way, the pouch and fluid absorbing bodies may be left inside the patient's uterus, and may degrade naturally after the hemostasis state has been achieved. Examples of bioabsorbable materials include, but are not limited by, polylactic acid (PLA), polyglycolic acid (PGA), oxidized cellulose, polycaprolactone (PCL), polydioxanone (PDO), trimethylene carbonate (TMC), polyvinyl alcohol (PVA), polyhydroxyalkanoates (PHAs), polyamides, polyethers, copolymers thereof, and/or and combinations thereof.

As used herein, the terms “bioabsorbable” or “biodegradable” refer to materials that are degraded by the body's enzymatic and/or hydrolytic pathways through a reaction against “foreign” material. Depending on the chemical nature of the material, the bioabsorbable material may degrade or absorb into the in vivo environment after a defined period, which can vary, for example, from a few hours to several months.

It should be appreciated that any and/or all components of the uterine hemostasis devices described here (at least the components which are inserted internally to the patient) may be formed of a biocompatible material. The term “biocompatible materials” used herein refers to materials that have the ability to perform with an appropriate host response in a specific application. Biocompatible materials have the quality of not having toxic or injurious effects on biological systems.

It should be appreciated that as with most other uterine hemostasis devices which are configured to absorb bodily fluids, the uterine hemostasis devices of the present disclosure may not be re-usable. To reduce the risk of infection, abscess formation, hematoma, wound dehiscence, mediastinitis, allergic reactions, adhesion formations, foreign body reactions, seromas, and/or any other unintended side effects, the fluid absorbing bodies of the present disclosure are configured to be appropriate discarded after use. Of course, in some embodiments, the applicator may be formed of a sterilizable material for re-use.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

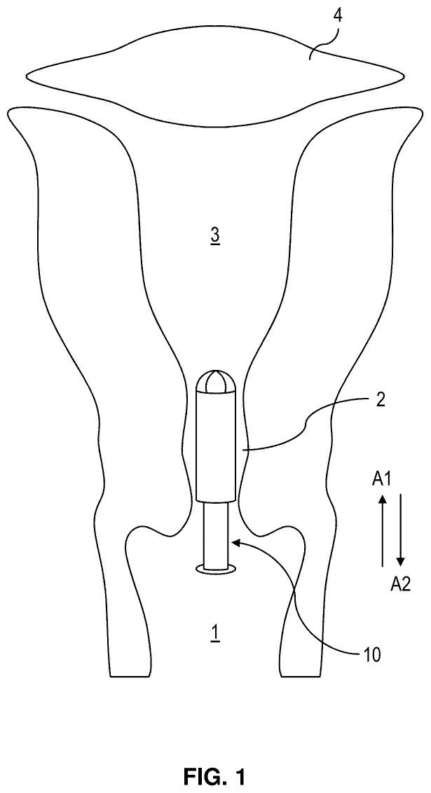

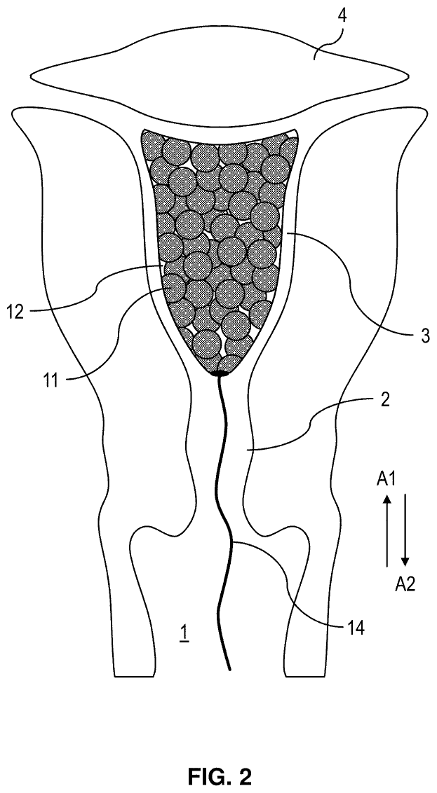

FIGS. 1-2 depict a process of deploying a uterine hemostasis device according to the present disclosure. A clinician may insert a uterine hemostasis device 10 along an axial direction A1, through a vagina 1 of a patient and subsequently position the system in the cervical canal 2. The uterine hemostasis device 10 may then be deployed to release one or more fluid absorbing bodies 11 into the uterus 3. The bodies 11 may absorb any fluids (e.g., blood) in the uterus 3 and subsequently expand to apply pressure to the walls of the uterus 3 and uterine fundus 4, as shown in FIG. 2. In this way, the bodies 11 may facilitate hemostasis in the patient's uterus 3, which may reduce the risk of hemorrhage.

In some embodiments, as shown in FIG. 2, the bodies 11 may be contained within a flexible pouch 12 which may be able to expand along with the bodies 11 as they absorb blood and other fluids from the uterus. The pouch may be accessible to a clinician via one or more tethers 14, which may be dangling or otherwise available in the vagina. In this way, the clinician may readily grasp the tethers and extract the system (e.g., pouch 12 enclosing the fluid absorbing bodies 11) from the uterus 3 by pulling the tethers 14 along the axial direction A2.

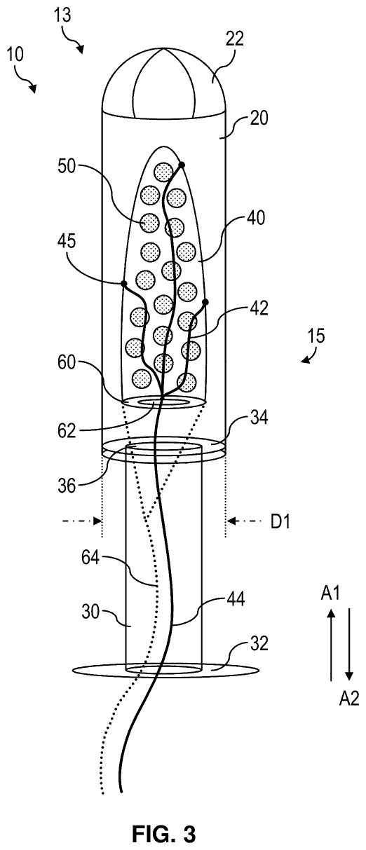

FIG. 3 shows a uterine hemostasis device 10 according to some embodiments. The system may include an applicator 15, which may serve to deliver a pouch 40 enclosing fluid absorbing bodies 50 to a patient's uterus. The applicator 15 may have a substantially hollow barrel 20, which may hold the pouch 40 and bodies 50 prior to deployment. A plunger 30, positioned coaxially to the barrel 20, may be moved in an axial direction (e.g., direction A1, as shown in FIG. 3) to expel the pouch 40 from the barrel 20. In some embodiments, the plunger 30 may include a first flange 32, which may serve as a push surface to a clinician, such that they may apply a force to the flange 32 to deploy the pouch 40 from the barrel 20. The plunger 30 may also include a second flange 34, in some embodiments, which may abut against the pouch 40 to displace the pouch out of the applicator. Accordingly, the second flange 34 may be sized appropriately relative to the pouch, such that the second flange 34 may displace the pouch within the barrel 20. As will be described in further detail below, in some embodiments, the plunger 30 may be hollow, with an inner channel 36 extending from the first flange 32 to the second flange 34.

In operation, a clinician may insert the device 10 inside a patient's cervical canal and subsequently apply force to the flange 32 to deploy the pouch 40 in the patient's uterus. Application of force to the flange 32 may move the plunger 30 and the second flange 34 through the barrel 20, thus displacing the pouch 40 out of the barrel 20. In some embodiments, a distal end 13 of the barrel 20 may have a valve 22, which may allow the pouch 40 to exit the barrel 20. The valve 22 may keep the pouch 40 and fluid absorbing bodies 50 in a sterile environment prior to deployment. In some embodiments, the valve 22 may be pressure-sensitive, such that it may be opened to permit the pouch 40 to exit the barrel 20 if sufficient pressure is applied. In some embodiments, the valve 22 may be a one-time valve (e.g., a frangible valve, such as a seal), although other embodiments of the valve 22 are also contemplated. In some embodiments, instead of having a valve, the distal end 13 of the barrel is an opening, such that the pouch 40 may freely exit the barrel 20 without having to pass any valve.

In some embodiments, the pouch 40, which may be formed of a flexible and/or expandible material, may contain the fluid absorbing bodies 50. For example, the pouch may be formed of a non-stretchable material (e.g., a non-stretchable open-worked mesh which may be scrunched or otherwise compressed in the applicator prior to deployment. It may therefore be permitted to relax once released in the uterus (and upon expansion of the fluid absorbing bodies), without physically stretching. In other embodiments, the pouch may be formed of a flexible material, such that it may stretch or otherwise expand in the uterus to accommodate the fluid absorbing materials. It should be appreciated that any suitable biocompatible material may be employed for the pouches of the present disclosure, and that the pouches may not significantly inhibit the expansion of the enclosed fluid expansion bodies.

The bodies 50 may be formed of a fluid absorbing material, and may, in some embodiments, expand upon absorption of one or more fluids (e.g., blood). Accordingly, prior to deployment, the bodies 50 may be sufficiently small to fit within the barrel 20. In some embodiments, the bodies may be pre-loaded with a therapeutic agent, such as a hemostatic agent. It should be appreciated that although multiple bodies 50 are shown in FIG. 3, embodiments in which a single fluid absorbing body is employed are also contemplated. As such, the present disclosure is not limited by the number of fluid absorbing bodies employed in the pouch. It should also be appreciated that although the bodies are shown to be substantially uniform in shape and size, the pouch may enclose any suitable number of different fluid absorbing body types, as the present disclosure is not so limited.

As shown in FIG. 3, in some embodiments, the pouch 40 may include one or more retrieval tethers 64. The retrieval tethers 64 may be connected to the pouch 40 in any suitable manner (e.g., welding, adhesives, sewing, tacking, etc.), and may extend through the channel 36 of the hollow plunger 30, such that they may be accessible to the clinician from the patient's vagina 1 (as shown in FIG. 2) and/or outside the vaginal opening. In operation, the clinician may pull the retrieval tethers 64 along an axial direction (e.g., axial direction A2) to extract the pouch 40 from the patient.

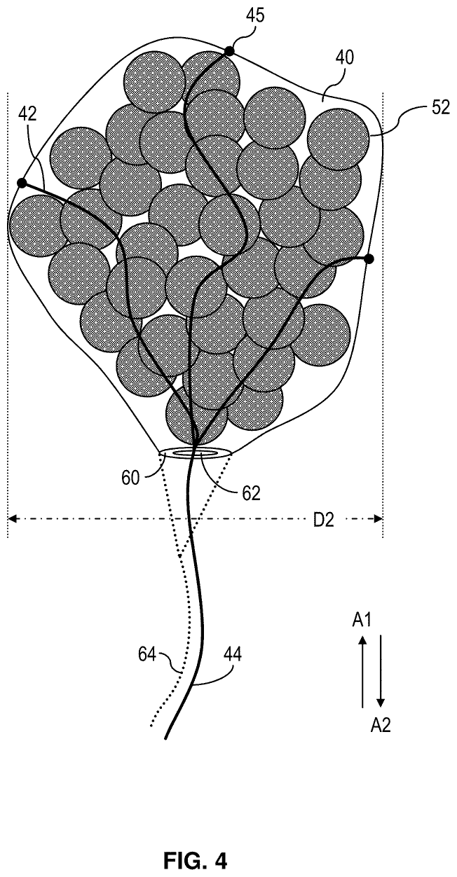

As shown in FIG. 4, in some embodiments, the pouch 40 may also include a ring 60. In some embodiments, the ring may be more rigid than the pouch. In some embodiments, the ring 60 may be connected to the retrieval tether 64. The ring 60 may be sized to be slightly larger than the cervical canal (e.g., the internal orifice of the cervical canal). As such, the ring 60 may keep the pouch 40 within the uterus during the hemostasis process. As will be described in greater detail below, the ring 60 may be sufficiently flexible (and/or collapsible) such that the ring may be extracted through the cervical canal with the retrieval tethers 64. The ring 60 may initially be inserted past the cervical canal with the assistance of the rigid applicator 15, which may temporarily stretch the cervical canal during delivery of the pouch 40. For example, the applicator may have a diameter D1 measured normal to its axial dimension, which may be slightly larger than a cross-section of the cervical canal. Of course, embodiments where the applicator diameter D1 is less than the cross-sectional size of the cervical canal are also contemplated. In some embodiments, the ring 60 may be collapsed in the barrel 20, such that it may automatically expand to a size larger than the cervical canal upon deployment from the barrel 20.

As shown in FIGS. 3-5, in some embodiments, the pouch 40 may also include one or more compression tethers 42, coupled to an internal surface of the pouch 40 at one or more locations 45. The compression tethers 42 may be connected to one another (if more than one tether is employed) through an inner opening 62 of the ring, connecting to at least one compression tether handle 44 extending through the channel 36 of the hollow plunger 30, as shown in FIG. 3. As will be described in further detail below, the compression tethers 42 may allow a clinician to compress the pouch after the hemostasis procedure to allow the pouch to be extracted from the patient's uterus.

FIG. 4 shows a deployed pouch of a uterine hemostasis device according to some embodiments. In operation, a clinician may have deployed the pouch 40 out of an applicator and into a uterus of a patient. The pouch 40, which may be flexible and/or expandible, may contain one or more fluid absorbing bodies, which may absorb fluids (e.g., blood) from the uterus and expand. It should be appreciated that the pouch 40 may be fluid permeable, such that the bodies may have access to blood within the uterus without having to exit the pouch. The expanded bodies 52 (which may be larger in volume than the bodies 50 shown in FIG. 3, due to the absorption of fluid), may therefore expand the pouch 40 and apply pressure to the uterine walls, providing a tamponing function to induce hemostasis. Accordingly, a diameter D2 of the pouch 40 measured normal to the axial direction (e.g., axial direction A1) may be greater than the applicator diameter D1, shown in FIG. 3.

It should be appreciated that although the expanded size of the pouch 40 (and accordingly, the expanded fluid absorbing bodies 52) may be useful in applying pressure to the uterine walls, the pouch and expanded fluid absorbing bodies may be too large to easily extract from the uterus after hemostasis. Thus, the compression tethers 42 may be used to initially compress the pouch 40 (and bodies 52), facilitating extraction from the patient.

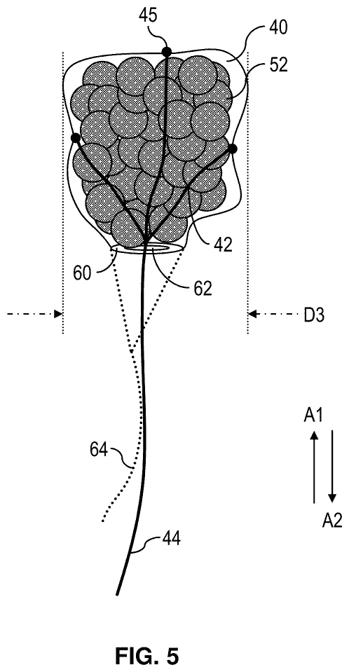

FIG. 5 shows a compressed pouch 40 according to some embodiments. The pouch 40 may be compressed by a clinician applying tension to one or more compression tether handles 44 in an axial direction (e.g., direction A2). The tether handle 44 may be connected to one or more compression tethers 42, which may in turn be connected to the pouch 40 (e.g., on an internal surface of the pouch) at one or more locations 45, such that pulling the tether handle 44 may compress the pouch 40 relative to the uterus. For example, the pouch 40 may be reduced in size such that its diameter D3 may be less than the diameter D2 of the pouch shown in FIG. 4.

In some embodiments, the compression tethers 42 may be formed of a flexible material, such that they may stretch to accommodate the compression of the pouch 40. In other embodiments, the compression tethers 42 may be substantially non-stretchable. Of course, any suitable biocompatible material which may be able to compress the pouch 40 at one or more locations 45 may be employed for the compression tethers.

It should be appreciated that handle 44 may be inhibited from extracting the pouch 40 from the uterus due to the presence of a ring 60, positioned against the cervical canal. As noted previously, the ring may be slightly larger than the cervical canal, such that it may inhibit the passage of the pouch out of the uterus. Accordingly, pulling on the tether handle 44 may only serve to compress the pouch 40, such that the pouch may be easier to extract. It should be appreciated that in some embodiments, the compression process may squeeze or compress the bodies 52, such that a small volume of the absorbed fluid (e.g., blood) may be expelled back into the uterus.

The clinician may then apply tension or pull a retrieval tether 64, connected to the pouch 40 and ring 60, to urge the ring 60 out of the uterus and through the cervical canal. In this way, the extraction process may be facilitated, as the size of the pouch may be reduced to limit the amount of pressure the pouch may apply to the cervical canal as the pouch is extracted. The retrieval tether 64 may be configured to collapse or otherwise deform the ring 60 to allow the ring to pass through the cervical canal. In some embodiments, the ring may be hinged, such that pulling the retrieval tether may open the hinges of the ring, such that it may more comfortably fit through the cervical canal.

In some embodiments, the retrieval tethers 64 and compression tether handles 44 may have unique characteristics to allow a clinician to differentiate between the two (or more, if multiple tethers are employed). For example, the retrieval tether 64 may be colored differently than the compression tether handle 44. In another example, the retrieval tether 64 may have a different texture or feel than the compression tether handle 44, such that the clinician may differentiate between the two without having visibility (e.g., during cases with massive blood loss). In yet another example, the tethers may have identifying labels at the ends that a clinician interacts with. Of course, other means of differentiating the tethers are also contemplated, as the present disclosure is not so limited.

In some embodiments, the uterine hemostasis devices described herein may not include any compression tethers. Accordingly, the size of the pouch may be reduced solely by the retrieval tether and pressure from the cervical canal.

It should be appreciated that all of the expanded bodies 52 may be simultaneously extracted from the uterus at once with the pouch 40. In this way, the risk of leaving behind a fluid absorbing body (which may pose a risk of infection) may be greatly reduced. In addition, the extraction process may be rendered easier for the clinician—such that they may not need to reach inside the patient to retrieve each individual fluid absorbing body, and more comfortable for the patient.

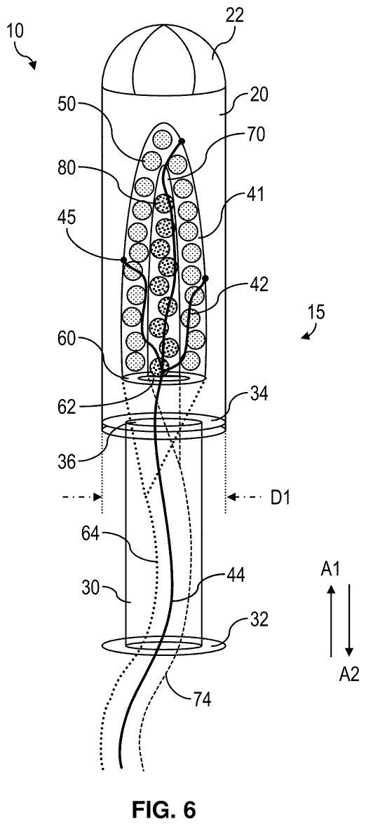

FIGS. 6-8B depict another embodiment of a uterine hemostasis device 10, which may function similarly to the system described in FIGS. 3-5, with the addition of a secondary pouch 70. In some embodiments, the secondary pouch 70 may be positioned inside a main pouch 41, which may also hold one or more fluid absorbing bodies 50. The secondary pouch may also include one or more fluid adsorbing bodies 80. The secondary pouch 70 may serve to further reduce the size of the main pouch 41 to make the extraction process of the main pouch 41 more comfortable. In some embodiments, the secondary pouch 70 may be separately extracted from the main pouch 41, such that the remaining volume of the main pouch 41 may more comfortably be extracted from a patient.

In operation, an applicator 15 may be used to deliver the main pouch 41 into the patient's uterus. A clinician may position a barrel 20 of the applicator 15 in the patient's cervical canal, and subsequently apply pressure to a flange 32 (e.g., in an axial direction A1, as shown in FIG. 6) to urge the main pouch 41 out of the barrel and into the uterus. Once the fluid absorbing bodies 50, 80 enter the uterus (via pouches 41, 70), they may initiate the hemostasis process by absorbing blood, expanding in size, and subsequently applying pressure to the uterine walls. It should be appreciated that both pouches 41 and 70 may be fluid permeable, such that the fluid absorbing bodies 50, 80 may have access to the uterine fluids without having to exit the pouches.

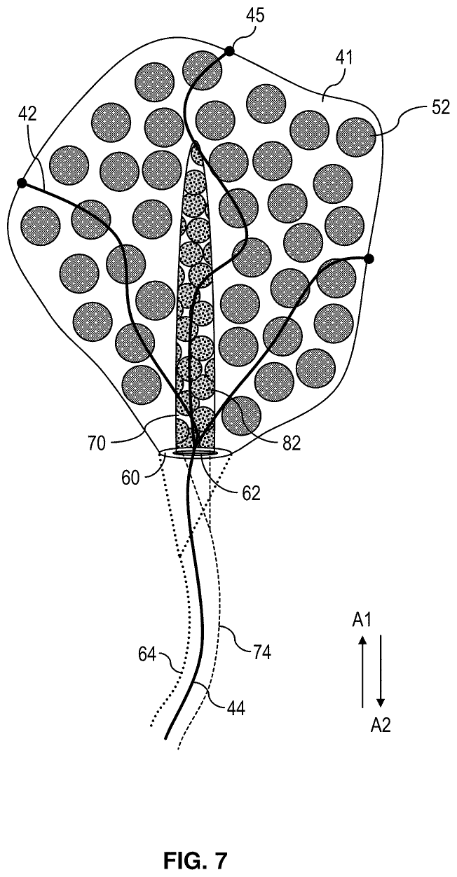

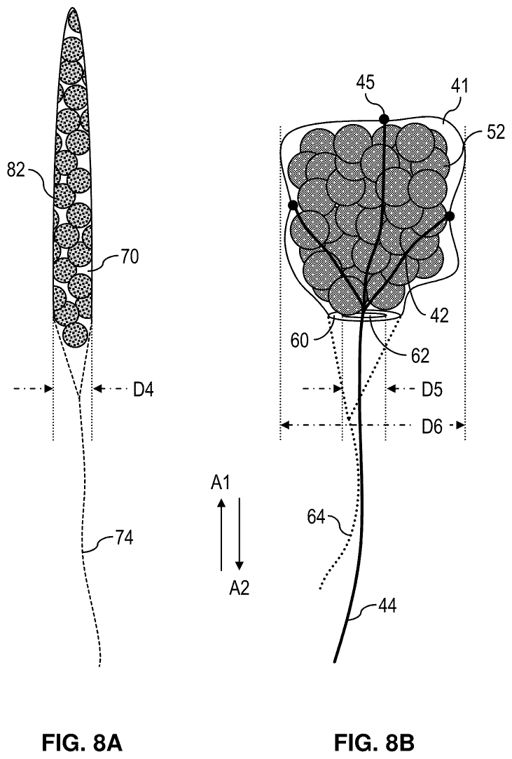

As shown in FIG. 7, after deployment in the uterus, the fluid absorbing bodies of pouches 41, 70 may expand and absorb fluid (e.g., blood) from the uterus, resulting in expanded bodies 52, 82. In some embodiments, the secondary pouch 70 may be less flexible than the main pouch 41. As such, the secondary pouch 70 may not expand as much as the main pouch 41. In other words, the size of the secondary pouch 70 in the applicator (see applicator 15 in FIG. 6) may be substantially similar to the size of the secondary pouch 70 in the uterus (see FIG. 7). For example, as shown in FIG. 8A, the diameter D4 of the secondary pouch 70 may be substantially similar in the pre-deployment stage (FIG. 6) and the post-deployment stage (FIG. 7).

In contrast, and as discussed previously, the main pouch 41 may expand to accommodate the expanding fluid absorbing bodies 50. The lack of flexibility of the secondary pouch 70 may inhibit the secondary pouch 70 from being too large, and subsequently, may reduce the need for the secondary pouch 70 to be compressed. In other words, the secondary pouch 70 may not need to be compressed to be comfortably extracted. As such, the secondary pouch diameter D4 may be smaller than the inner diameter D5 of the ring 60 (e.g., the size of the opening 62). Accordingly, the secondary pouch 70 may be able to be extracted from the uterus through the ring 60 without any compression. As discussed in relation to the retrieval tethers 64 connected to main pouch 41, in some embodiments, the secondary pouch 70 may be extracted from the uterus by a clinician pulling on one or more retrieval tethers 74 (e.g., along an axial direction A2).

Following deployment, the pouches 41, 70 may be left in the uterus for a sufficient period of time to allow for hemostasis to occur, during which the fluid absorbing bodies 50, 80 may absorb blood from the uterus and expand, applying pressure to the uterine walls. The clinician may then elect to first extract the secondary pouch 70 with retrieval tethers 74, reducing the overall volume of the main pouch 41. Subsequently, the clinician may follow a similar protocol as described with reference to FIGS. 3-5, including first compressing the main pouch 41 using one or more compression tethers 42 (operated with a compression tether handle 44), and then extracting the compressed main pouch 41 from the uterus. It should be appreciated that the removal of the secondary pouch 70 may reduce the overall volume of the main pouch 41, such that the compressed diameter of the main pouch 41 in FIG. 8B (see diameter D6) may be lower than the compressed diameter of the main pouch 41 in FIG. 5 (see diameter D3). Of course, embodiments in which the sizes of the main pouch 41 are not affected by the secondary pouch are also contemplated.

In some embodiments, the bodies 50 may be similar to bodies 80, in terms of material composition and structure (e.g., porosity, shape, etc.), whereas in other embodiments, the bodies 50 may be different from bodies 80. It should be appreciated that the present disclosure is not limited by the type, arrangement, variation, size, structure, and/or any other parameter of the fluid absorbing bodies (contained in either main pouch 41, 70).

It should be appreciated that although only one secondary pouch is shown in the figures, in some embodiments, multiple secondary pouches, each with a respective retrieval tether, may be employed to reduce the size of the main pouch 41 in a step-wise fashion.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

1. A uterine hemostasis device comprising:
at least one pouch enclosing at least one fluid absorbing body, wherein the at least one fluid absorbing body is configured to expand in volume upon absorption of a fluid; and
at least one tether operatively coupled to an internal surface of the at least one pouch,
wherein the at least one tether is configured to be manipulated, and wherein manipulation of the at least one tether at least partially compresses the at least one fluid absorbing body.

2. The uterine hemostasis device of claim 1, wherein the at least one tether is coupled to a plurality of locations on the internal surface of the at least one pouch.

3. The uterine hemostasis device of claim 1, wherein the at least one pouch is formed of a fluid-permeable material.

4. The uterine hemostasis device of claim 1, wherein the at least one fluid absorbing body comprises a plurality of fluid absorbing bodies.

5. The uterine hemostasis device of claim 4, wherein the plurality of fluid absorbing bodies comprises gauze.

6. The uterine hemostasis device of claim 1, wherein the at least one fluid absorbing body is formed at least partially of a hemostatic agent.

7. The uterine hemostasis device of claim 1, further comprising an applicator configured to deliver the at least one pouch to a patient's uterus.

8. The uterine hemostasis device of claim 1, further comprising a ring, wherein the manipulation of the at least one tether at least partially compresses the at least one fluid absorbing body against the ring.

9. The uterine hemostasis device of claim 8, wherein the ring is connected to the at least one pouch.

10. The uterine hemostasis device of claim 8, wherein the at least one tether passes through the ring.

11. The uterine hemostasis device of claim 1, further comprising at least one retrieval tether operatively coupled to the at least one pouch, wherein the at least one retrieval tether is configured to be manipulated to displace the at least one pouch.

12. The uterine hemostasis device of claim 11, further comprising a ring, wherein the ring is connected to the at least one retrieval tether.

13. The uterine hemostasis device of claim 1, further comprising a second pouch containing a second at least one fluid absorbing body, the second pouch disposed at least partially in the at least one pouch.

14. A uterine hemostasis device comprising:
a first pouch containing a first at least one fluid absorbing body;
a second pouch containing a second at least one fluid absorbing body, the second pouch disposed at least partially in the first pouch;
a first tether operatively coupled to the first pouch; and
a second tether operatively coupled to the second pouch.

15. The uterine hemostasis device of claim 14, wherein the first pouch and the second pouch formed of a fluid-permeable material.

16. The uterine hemostasis device of claim 14, further comprising a third tether operatively coupled to at least one of the first pouch and the second pouch, wherein the third tether is configured to compress at least one of the first and second at least one fluid absorbing body.

17. The uterine hemostasis device of claim 14, wherein at least one of the first and second at least one fluid absorbing body is formed at least partially of a hemostatic agent.

18. The uterine hemostasis device of claim 14, further comprising an applicator configured to deliver the first pouch and the second pouch to a patient's uterus.

19. (canceled)

20. The uterine hemostasis device of claim 14, wherein the first tether is coupled to an internal surface of the first pouch.

21. (canceled)

22. The uterine hemostasis device of claim 14, wherein the second tether is coupled to an internal surface of the first pouch.

23. (canceled)

24. The uterine hemostasis device of claim 14, further comprising a ring, wherein manipulation of the first tether compresses the first pouch against the ring.

25. The uterine hemostasis device of claim 14, further comprising a ring, wherein manipulation of the second tether compresses the second pouch against the ring.

26. A method of operating a uterine hemostasis device comprising:
positioning a first pouch in a uterus of a patient, the first pouch containing a first at least one fluid absorbing body;
absorbing fluids from the uterus of the patient with the first at least one fluid absorbing body; and
manipulating a first tether to at least partially compress the first at least one fluid absorbing body, wherein the first tether is coupled to an internal surface of the first pouch.

27-32. (canceled)

33. A method of operating a uterine hemostasis device comprising:
positioning a first pouch and a second pouch in a uterus of a patient, the first pouch containing a first at least one fluid absorbing body and the second pouch containing a second at least one fluid absorbing body, wherein the second pouch is disposed at least partially within the first pouch;
absorbing fluids from the uterus of the patient with the first at least one fluid absorbing body and the second at least one fluid absorbing body;
extracting the second pouch from the first pouch and from the uterus of the patient; and
extracting the first pouch from the uterus of the patient.

34-39. (canceled)
